(19) United States
(12) Patent Application Publication
(10) Pub. No.: US 2023/0233439 A1
(43) Pub. Date: Jul. 27, 2023

(54) METHOD FOR COSMETIC TREATMENT BY COMBINED ILLUMINATION AND APPLICATION OF A COMPOSITION COMPRISING NIACINAMIDE, AND ASSOCIATED DEVICE

(71) Applicant: LIGHTINDERM, Paris (FR)

(72) Inventors: Lieve Declercq, Destelbergen (BE); Cyprien Guermonprez, Paris (FR)

(73) Assignee: LIGHTINDERM, Paris (FR)

(21) Appl. No.: 17/918,986

(22) PCT Filed: May 4, 2021
(86) PCT No.: PCT/EP2021/061729

§ 371 Date: Oct. 14, 2022

(30) Foreign Application Priority Data
May 6, 2020 (FR) 2004500

(51) Int. Cl. A61K 8/67; A61K 8/66; A61Q 19/00

(52) U.S. Cl. CPC A61K 8/675; A61K 8/66; A61Q 19/00; C12Y 401/99003; A61K 2800/10; A61K 2800/81

(57) ABSTRACT

The non-therapeutic cosmetic treatment method includes at least: —applying an active composition including at least niacinamide onto the skin of a subject, and—illuminating the skin using at least one light source emitting at a wavelength of between 620 and 690 nanometres, preferably 660 nanometres, the illumination being carried out before, at the same time as or after the application of the composition. A device is configured for implementing such a method.

### TECHNICAL FIELD

The invention falls within the field of nontherapeutic cosmetic treatments by illuminating the skin. The invention also falls within the field of cosmetic treatments by applying a composition comprising at least one active agent.

The invention also relates to a device for performing such a process.

### PRIOR ART

To limit the effects of aging on bodily or facial skin, such as wrinkles, the density of the dermis, and the luminosity and uniformity of the complexion, it is known practice to apply cream-type compositions including one or more active agents. These compositions usually have a limited effect but, above all, the effects only become apparent after a more or less long treatment period.

It is known practice to combine the application of a composition with illumination to accelerate the effect of the active agents applied to the skin.

By way of example, US 2008/031833 relates to a process for improving the condition of the skin and for delaying and/or preventing the signs of its aging. This process consists in applying a composition whose active agent is chosen from a multitude of active care agents, and of an illumination which may be in continuous or pulsed mode, and which scans the UV spectrum, the visible spectrum and the infrared spectrum. However, said publication does not mention any significant results as regards attenuating skin aging.

US 2007/112042 describes the combination of the application of a topical vasodilator and illumination with a pulsed laser at 585 nm to increase collagen production. The results are not reported either.

US 2009/209600 discloses a device for applying a product to improve skin moisturization using a cosmetic composition formulated from niacinamide, the product being dispensed by a heating device consisting of an infrared lamp.

EP 2861203 discloses a process for the cosmetic treatment of greasy skin, in which the skin is subjected to a quasi-chromatic light exposure in the range from 300 to 700 nm, and optionally to a second light exposure in the red and infrared range. The simultaneous application of a composition based on hollow particles such as hydrophobic silica aerogel particles is mentioned. However, this combination acts specifically on sebum secretion.

US 2018/200174 discloses a cosmetic treatment process acting notably as an antiaging treatment, in which an algal or plant extract comprising a light-activatable enzyme, a baobab seed extract, a Tephrosia purpurea extract and a prebiotic or probiotic extract is applied to the skin, supplemented by illumination in a range from 630 to 700 nm and/or a range from 420 to 460 nm, the latter range interacting more particularly with the enzyme.

WO 2016/146778 discloses an electroluminescent skin treatment device, and an associated process, acting notably as an antiaging device, and including illumination in three combined wavelength ranges: 510-536 nm, 650-670 nm and 768-792 nm. The illumination may be supplemented by the application of an active agent.

EP 3586926 discloses an antiaging skin treatment process, in which the application of glucosamine hydrochloride is combined with illumination in the range from 600 nm to 750 nm and/or from 750 nm to 1000 nm.

US 2014/236265 discloses an antiacne process in which a cleansing composition is applied to the skin, followed by illumination in the range from 390 to 430 nm, this illumination being followed by the application of an antioxidant composition to limit the adverse effects on the skin of the phototherapy. In one particular embodiment, additional illumination in the range from 600 to 700 nm is envisaged.

### OBJECT OF THE INVENTION

There is thus a need for a method for the cosmetic treatment of the skin which combines the effects of illumination and one or more active agents to significantly reduce the effects of skin aging.

To this end, the invention is directed toward a treatment method and an associated device which demonstrably and visibly attenuate the main effects of skin aging, notably wrinkles, loss of density of the dermis, reduction in the luminosity of the complexion and lack of uniformity of the complexion.

### DISCLOSURE OF THE INVENTION

To this end, the invention is directed toward a nontherapeutic cosmetic treatment process, comprising at least the steps of:
applying to the skin of an individual an active composition comprising at least niacinamide, and
illuminating the skin with at least one light source emitting at a wavelength of between 620 and 690 nanometers,
   the illumination being performed before, simultaneously with or after the application of the composition.

Contrary to the processes disclosed in the documents cited in the prior art, the specific combination of niacinamide and illumination at a wavelength of between 620 and 690 nanometers, more preferentially 660 nanometers, made it possible to demonstrate the antiinflammatory effect of the process of the invention on keratinocytes and the increase in procollagen-1 production, thereby attesting to an antiaging effect on the skin.

The cosmetic treatment method may also include the following optional features, considered in isolation or in any possible technical combination:—
the active composition also comprises photolyase,
the skin is also illuminated with at least one light source emitting at a wavelength of between 410 and 470 nanometers, preferably 440 nanometers,
   The combination of photolyase in the composition, in addition to niacinamide, and a wavelength of between 410 and 470 nanometers, more preferentially 440 nanometers, made it possible to observe, surprisingly, a reduction in the density of thymine dimers exposed to UV, and also a visible reduction in wrinkles, an increase in the density of the dermis, a smoothing effect, and uniformity of the complexion.
the illumination of the skin is also performed with at least one light source emitting at a wavelength of between 750 and 810 nanometers, preferably 780 nanometers,
the skin is also illuminated with at least one light source emitting at a wavelength of between 910 and 970 nanometers, preferably 940 nanometers,
the mass percentage of niacinamide in the composition is between 0.5% and 10%, preferably between 0.5% and 7%, more preferentially between 3% and 7%,
the mass percentage of photolyase in the composition is between 0.001% and 0.01%,
the active composition comprises sodium hyaluronate,
the active composition comprises a Laminaria algal extract,
the active composition comprises vitamin C,
the active composition comprises vitamin E,
the active composition also comprises acetyl hexapeptide-8,
the active composition also comprises caffeine,
the illumination is performed simultaneously with the application of light to the skin,
the illumination spectrum is discontinuous,
the illumination time is less than 180 seconds, preferably less than or equal to 30 seconds.

The invention also relates to a nontherapeutic cosmetic treatment device comprising at least means for applying to the skin of an individual an active composition comprising at least niacinamide, and means for illuminating the skin with at least one light source emitting at a wavelength of between 620 and 690 nanometers, the means for illuminating and applying the active composition being configured so that the illumination can be performed before, simultaneously with or after the application of the composition.

The device of the invention may also include the following optional features, considered in isolation or in any possible technical combination:—
the active composition also comprises photolyase,
the means for illuminating the skin also include at least one light source emitting at a wavelength of between 410 and 470 nanometers, preferably 440 nanometers,
the means for illuminating the skin also include at least one light source emitting at a wavelength of between 750 and 810 nanometers, preferably 780 nanometers,
the means for illuminating the skin also include at least one light source emitting at a wavelength of between 910 and 970 nanometers, preferably 940 nanometers.

Finally, the invention relates to a nontherapeutic cosmetic treatment process comprising the implementation of the abovementioned device.

### PRESENTATION OF THE FIGURES

Other features and advantages of the invention will emerge clearly from the description given below, by way of indication and not in any way limiting, with reference to the attached figures, among which:

FIG. 1 is a histogram illustrating the percentage of protection of cells by means of measuring the interleukin-6 production, the cells having been previously exposed to a combination of UVA and UVB and treated by illumination alone at a wavelength of 660 nanometers, by the application of niacinamide alone, and by the treatment process of the invention with a composition including niacinamide and discontinuous illumination at a wavelength of 660 nanometers according to 10 different illumination programs.

FIG. 2 is a histogram illustrating the percentage protection of cells by measuring the interleukin-8 production, the cells having been previously exposed to a combination of UVA and UVB and treated by illumination alone at a wavelength of 660 nanometers, by the application of niacinamide alone, and by the treatment process of the invention with a composition including niacinamide and discontinuous illumination at a wavelength of 660 nanometers according to 10 different illumination programs, by illumination alone at a wavelength of 660 nanometers.

FIG. 3 is a histogram illustrating the percentage stimulation of procollagen-1 production of cells previously exposed to a combination of UVA and UVB and treated by illumination alone at a wavelength of 660 nanometers, by the application of niacinamide alone, and by the treatment process of the invention with a composition including niacinamide and discontinuous illumination at a wavelength of 660 nanometers according to two different illumination programs, and by illumination alone at a wavelength of 660 nanometers.

FIG. 4 is a histogram illustrating the percentage reduction in the area of thymine dimers of skin explants previously exposed to a combination of UVA and UVB, without treatment, treated by application of a composition comprising niacinamide and photolyase alone, and treated by the treatment process of the invention with a composition including niacinamide and photolyase, and discontinuous illumination at wavelengths of at least 660 nanometers and 440 nanometers according to three different programs.

FIG. 5 illustrates two photographs of a skin explant showing the damage through the presence of thymine dimers from exposure to UVA and UVB, the explant in the photograph on the left having been subjected to no treatment, and the explant in the photograph on the right having been subjected to the treatment process of the invention with a composition including niacinamide and photolyase, and discontinuous illumination at a wavelength of at least 660 nanometers and 440 nanometers.

FIG. 6 illustrates six ultrasound images 6a, 6b, 6c, 6d, 6e, 6f, respectively, illustrating the density of the dermis of two individuals who have been treated for 28 days via the treatment method of the invention with a composition including niacinamide and photolyase and discontinuous illumination at a wavelength of at least 660 nanometers and 440 nanometers, in comparison with the first day of treatment and the 28th day of treatment with a placebo.

FIG. 7 is a histogram schematically comparing the average percentage reduction in non-echogenic skin area for all the treated individuals at 28 days of treatment, and for each of whom one half of the face is treated via the treatment method of the invention with a composition including niacinamide and photolyase and discontinuous illumination at a wavelength of at least 660 nanometers and 440 nanometers, in comparison with the other half of the face treated with a placebo.

FIGS. 8 and 9 are histograms schematically comparing the average percentage efficacy on average relief and maximum amplitude of a 3D profile by profilometry for all the treated individuals at 14 days and 28 days of treatment and for each of whom one half face is treated via the treatment method of the invention with a composition including niacinamide and photolyase and discontinuous illumination at a wavelength of at least 660 nanometers and 440 nanometers, in comparison with the other half of the face treated with a placebo.

FIG. 10 is a histogram schematically comparing the average percentage change in skin smoothness for all the treated individuals at 14 days and 28 days of treatment and for each of whom one half of the face is treated via the treatment method of the invention with a composition including niacinamide and photolyase and discontinuous illumination at a wavelength of at least 660 nanometers and 440 nanometers, in comparison with the other half of the face treated with a placebo.

FIG. 11 is a schematic representation in cross section of the device of the invention without its capsule for applying the composition.

FIG. 12 is a schematic representation in cross section of the device of the invention illustrated with the capsule for applying the composition, and

FIG. 13 is a schematic front view of an example of implementation of the electroluminescent device of the device of the invention of FIGS. 11 and 12.

### DETAILED DESCRIPTION OF THE INVENTION

The nontherapeutic cosmetic treatment process of the invention essentially comprises a step of applying to the skin of an individual an active composition comprising at least niacinamide as active agent and illuminating, preferably simultaneously, this same area of the skin with a light source emitting in the red spectrum at a wavelength of about 660 nm, considering a half-band width of 30 nm. When the illumination is performed before or after the application of the composition, a maximum time between the illumination and the application of the niacinamide of 30 minutes, preferably 15 minutes, is considered.

The cosmetic treatment process of the invention is applied exclusively to skin free of open wounds or of any other pathology. The process of the invention is applied to healthy skin, and more particularly to skin that shows signs of aging essentially due to its exposure to the sun.

in vitro tests of measuring the antiinflammatory effect on epidermal cells and the antiaging effect on these cells by the process of the invention were performed. The inflammation of the cells and the measurement of the antiaging effect are based on the irradiation of a cell culture with a combination of UVA and UVB. These cells are treated either with niacinamide alone, or with illumination at 660 nm alone, or with the combination of the application of niacinamide and illumination at 660 nm according to the process of the invention and according to the following protocol:
at time T−24 h, a treatment is applied, it being understood that this treatment is one of the three comparative treatments mentioned previously,
at time T0, UVA and UVB irradiation is performed, followed by the same treatment as at T−24 h,
at time T+24 h, the same treatment is applied, and
at T+48 h, the supernatant is collected.

Pure niacinamide at a concentration of 0.4 mg/mL or 1.2 mg/mL is used.

For the treatments inducing illumination at 660 nm, 10 different programs are evaluated. These programs are defined in Table 1 below.

TABLE 1
Parameters associated with each illumination program
With reference to the parameters indicated in the above table, the following
clarifications are made. The irradiation performed is in pulsed mode and has a
rectangular signal. The period T of the signal corresponds to the sum of the period Tₒₙ
during which the signal is at its maximum, and of the period T_{off} during which the signal
is zero. The irradiance corresponds to the power density per unit area during the period
Tₒₙ. The fluence is the amount of energy per unit area during the period Tₒₙ. The duty
cycle corresponds to the ratio between the period Tₒₙ and the period T.

| Program | 1 | 2 | 3 | 4 | 5 | 6 | 7 | 8 | 9 | 10 |
|---|---|---|---|---|---|---|---|---|---|---|
| Irradiance | 4.0 | 26.6 | 10.6 | 10.6 | 2.7 | 10.6 | 10.6 | 5.3 | 10.6 | 10.6 |
| (mW/cm²) | | | | | | | | | | |
| Duty | 50 | 50 | 50 | 50 | 50 | 20 | 71.4 | 50 | 50 | 50 |
| cycle (%) | | | | | | | | | | |
| Period T | 14 | 14 | 70 | 350 | 14 | 15 | 14 | 14 | 14 | 14 |
| (ms) | | | | | | | | | | |
| Fluence | 30 | 199 | 80 | 80 | 80 | 32 | 114 | 80 | 80 | 199 |
| (mJ/cm²) | | | | | | | | | | |
| Time (s) | 15 | 15 | 15 | 15 | 60 | 15 | 15 | 30 | 15 | 37.5 |

On conclusion of the protocol defined previously, three types of supernatants are obtained: the first treated only with niacinamide, the second subjected to illumination according to one of the 10 illumination programs at 660 nm, and the third subjected to the combination of niacinamide and illumination according to one of the 10 illumination programs at 660 nm.

Attention is primarily focused on the antiinflammatory effect of the process of the invention on epidermal cells, the keratinocytes. UV irradiation induces inflammation of the keratinocytes. The reason for this is that UV rays are highly energetic rays that are capable of producing free radicals in the cells by breaking covalent bonds between molecules. Free radicals are reactive and induce an inflammatory cascade characterized by the production of proinflammatory proteins (p53, pro-opiomelanocortin (POMC), interleukins, etc.) which can be measured in the cell supernatant.

The common cytokines with a broad spectrum of inflammations are the interleukins 6 and 8 (IL-6 and IL-8), which are chosen as measurement parameters, it being understood that UVA and UVB induce damage notably on keratinocytes, which produce more interleukins 6 and 8 than do cells not exposed to UV.

According to the protocol, at T+48 h, the cell supernatant is collected and the concentrations of interleukins 6 and 8 are measured and compared to the concentrations of interleukins 6 and 8 produced by cells exposed to UV and not subjected to any of the three abovementioned treatments (UV control) and to untreated and non-irradiated cells (non-irradiated control). A degree of protection of the treated cells relative to the UV control is evaluated according to the following formula:

$$Protection ( \% ) = ( \frac{UV control average - Concentration IL 6 or IL 8}{UV control average - Non - irradiated control average} ) \times 100 \qquad [ Math . 1 ]$$

Non-irradiated cells show 100% protection and irradiated cells show 0% protection. A negative result indicates an increase in the concentration of IL-6 or IL-8 and a positive result indicates a decrease in the concentration of IL-6 or IL-8, illustrating protection of the cell or faster repair of the UV-induced damage. A protection level of 100% ideally corresponds to the total suppression of the deleterious effects of UV on the skin cells.

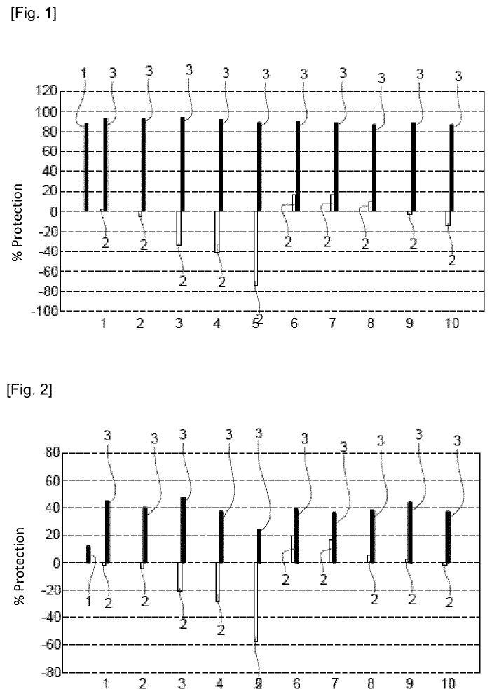

The results for interleukin-6 are shown in FIG. 1 and for interleukin-8 in FIG. 2.

In these figures, reference 1 corresponds to treatment with niacinamide alone at a concentration of 1.2 mg/ml, the references 2 correspond to treatment by illumination at 660 nm alone for each of the 10 illumination programs indicated in Table 1, and the references 3 correspond to treatment according to the process of the invention combining the effects of niacinamide at a concentration of 1.2 mg/ml and illumination at 660 nm for each of the 10 illumination programs indicated in Table 1.

It is seen that, irrespective of the illumination program, the process of the invention has a beneficial effect on the cells exposed to UV relative to the cells exposed to UV and/or which have been subjected only to niacinamide. These beneficial effects are particularly visible on the interleukin-8 concentration measurements. It is also seen that although some illumination programs associated with a treatment by illumination alone at 660 nm also increase the protection of the cells relative to the application of niacinamide alone, the process of the invention, for all the illumination programs, ensures a much greater protection of the skin cells than for these treatments of illumination alone at 660 nm.

Attention is now focused on skin aging, known as “photoaging”, induced by UV exposure. One of the parameters of cutaneous aging observed clinically is the appearance of wrinkles associated in part with a decrease in collagen production by the cells of the dermis, the fibroblasts. This decrease may be accompanied by an increased production of metalloproteases (MMP), which are responsible for the degradation of collagen fibers in the dermis. The production of procollagen-1 produced by fibroblasts is measured in the supernatants obtained from the previously defined protocol in which the cells were illuminated either without any other treatment or in combination with niacinamide at a concentration of 0.4 mg/ml, and compared with treatment with niacinamide alone at a concentration of 0.4 mg/ml.

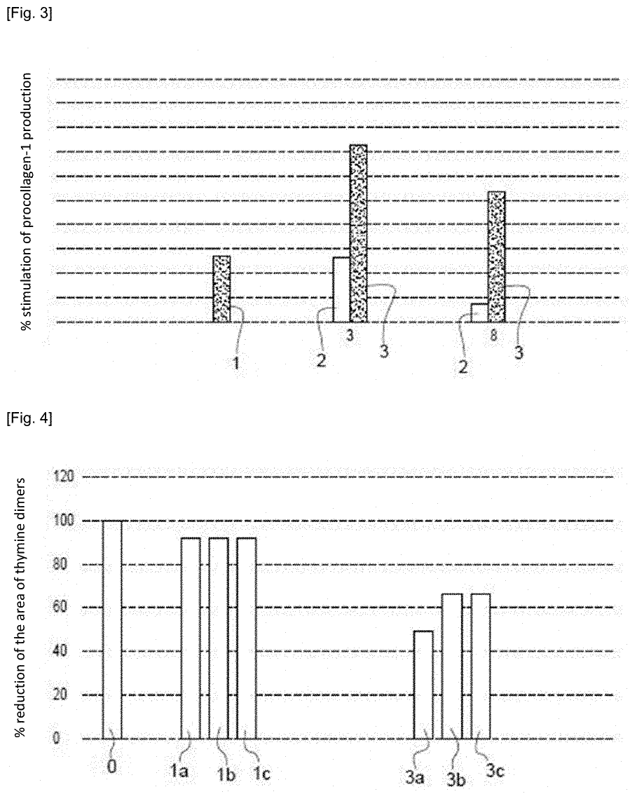

The results are shown in FIG. 3 for illumination programs 3 and 8. As in FIGS. 1 and 2, reference 1 corresponds to treatment with niacinamide alone, the references 2 correspond to treatment with illumination at 660 nm alone for illumination programs 3 and 8 shown in Table 1, and the references 3 correspond to a treatment according to the process of the invention combining the effects of niacinamide and illumination at 660 nm for illumination programs 3 and 8 shown in Table 1.

Increased production of procollagen-1 induced by the treatment via the process of the invention is observed, compared with a treatment with niacinamide, and treatment with illumination at 660 nm alone.

The results presented previously demonstrate the unexpected synergy between niacinamide and illumination at a wavelength of 660 nm on the antiinflammatory effect of the process on keratinocytes and the increase in the production of procollagen-1, thus illustrating the skin-protecting and antiaging effects of the cosmetic treatment process of the invention.

This combination between the application of niacinamide and illumination with “red” light at a wavelength centered on 660 nm, i.e. between 620 and 690 nanometers, is not demonstrated in the abovementioned prior art. This combination is of a synergistic nature with regard to its effects previously demonstrated with reference notably to FIGS. 1 to 3, which go beyond the sum of the effects of niacinamide on the one hand, and of illumination at 660 nm on the other hand. This synergy could not be made evident solely by the knowledge of niacinamide on the one hand and red light on the other hand, even though each of these two treatments was already known to have a beneficial effect on the skin in the context of processes involving other wavelengths and/or other active agents.

The cosmetic treatment process of the invention may also comprise the application of another active agent, photolyase, and additional illumination at a wavelength of 440 nm, considering a half-band width of 30 nm. Other wavelengths and other active agents may also be envisaged, as will be seen later.

in-vitro tests are performed for a treatment according to the process of the invention consisting of the application of a composition comprising niacinamide and photolyase, and illumination at 660 nm, 440 nm, and 780 nm or 940 nm. All these wavelengths are considered with a half-band width of 30 nm. Attention is more particularly focused on the addition of the combination of photolyase/illumination at 440 nm which results in a change in the three-dimensional structure of photolyase without any other modification of its chemical structure.

As regards the illumination applied during these tests, three different programs are evaluated. For each program, three wavelengths are applied together as shown in Table 2 below.

TABLE 2
Parameters associated with each illumination program

| | Program 1 | | Program 2 | | Program 3 | |
|---|---|---|---|---|---|---|
| | 30 seconds | | 15 seconds | | 15 seconds | |
| | Ir- | | Ir- | | Ir- | |
| | radiance | Fluence | radiance | Fluence | radiance | Fluence |
| | (mW/ | (mJ/ | (mW/ | (mJ/ | (mW/ | (mJ/ |
| λ | cm²) | cm²) | cm²) | cm²) | cm²) | cm²) |
| 440 | 3.8 | 57 | 3.8 | 29 | 7.4 | 56 |
| 660 | 5.3 | 80 | 5.3 | 40 | 10.6 | 80 |
| 780 | — | — | — | — | 4 | 30 |
| 940 | 1.5 | 23 | 1.5 | 11 | — | — |
| TOTAL | 10.6 | 159 | 10.6 | 80 | 22 | 165 |

Measurements of DNA damage by UV exposure are made by means of the quantification of thymine dimers. The protocol consists in observing and measuring the area of thymine dimers on skin explants exposed to UVA and UVB without treatment, with niacinamide and photolyase, with illumination as indicated in the three programs in Table 2 above, and with a combination of niacinamide and photolyase application and illumination as indicated in Table 2 above.

The results are shown in FIGS. 4 and 5.

In FIG. 4, reference 0 corresponds to a UV control which received no treatment, references 1a, 1b and 1c correspond to treatment with niacinamide alone according to, respectively, the illumination programs 1, 2 and 3 of Table 2, and references 3a, 3b and 3c correspond to treatment according to the process of the invention combining the effects of niacinamide and illumination according to, respectively, the illumination programs 1, 2 and 3 of Table 2.

With reference to FIG. 5, photograph 4 shows the presence and distribution of thymine dimers, the density of which reflects the amount of damage associated with exposure to UV light, this explant not having undergone any treatment. Photograph 5 shows the presence and distribution of thymine dimers in an explant treated via the process of the invention according to program 1.

The results presented show a significant reduction in the density of thymine dimers in a skin explant exposed to UV light and treated via the process of the invention, compared to treatment with niacinamide and photolyase and exposure to illumination alone. These results thus illustrate the ability of the process of the invention to repair cosmetic damage caused by UV exposure, and more generally, to regenerate the skin in order to improve its appearance.

Clinical tests were conducted on 33 women from 35 to 45 years old with phototypes I to III. For 28 days, these women received the treatment according to the process of the invention 6 days out of 7. The illumination corresponds to program 3 presented in Table 2. The composition applied includes, in mass percentages, 5% niacinamide (Niacinamide PC), 0.005% photolyase (Photosomes V), 0.0005% acetyl hexapeptide-8, 0.012% Laminaria algal extract, 0.1% sodium hyaluronate, 0.076% Caesalpinia spinosa fruit extract, 0.016% Kappaphycus alvarezii algal extract, 0.2% caffeine, 0.2% vitamin C and 0.2% vitamin E. The treatment is performed by simultaneous application of the composition and illumination according to program 3. The treatment according to the invention is performed on one half of the face on six areas from the chin to the forehead. The other half of the face is treated with a placebo, the composition of which is a moisturizing cream. The choice of the treated side is determined randomly. Each area is treated for 15 seconds according to program 3 in Table 3; the face treatment is thus completed in 3 minutes.

The effects of the treatment after 28 days, in comparison with the first day of treatment, on wrinkles, dermal density, smoothing effect and luminosity of the complexion are evaluated.

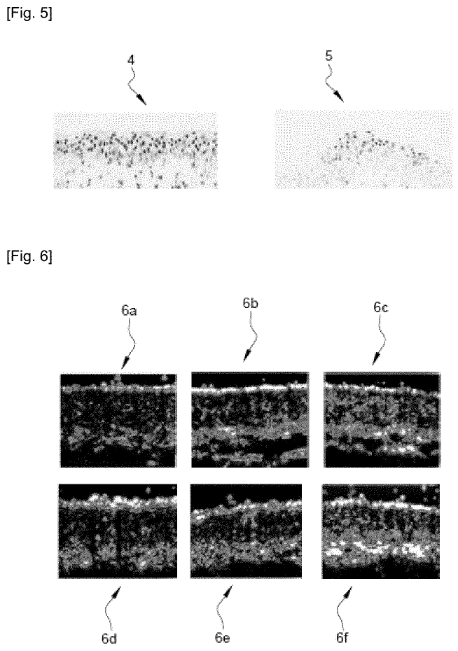

FIGS. 6 and 7 illustrate the results obtained regarding the dermal density. FIG. 6 represents six ultrasound measurements of the non-echogenic skin surface taken, respectively, on the first day of treatment (FIGS. 6a, 6d), on the 28th day of treatment via the process of the invention (FIGS. 6c, 6f) and on the 28th day of placebo treatment (FIGS. 6b, 6e) for two different individuals (6a, 6b, 6c; 6d, 6e, 6f).

It is seen that the area of the face treated via the process of the invention in the two representative individuals (FIGS. 6c, 6f) shows a significant reduction in the black surface corresponding to the non-echogenic surface, the hypoechogenic sub-epidermal band being a characteristic manifestation of skin aging in areas of chronic sun exposure. In addition, the number of high-density pixels increased substantially, indicating the reappearance of denser, echogenic dermal fibers. For the placebo treatment, in both individuals, the non-echogenic area and the number of high-density pixels did not change significantly.

FIG. 7 reports, in the form of a histogram, the average percentage reduction in the non-echogenic skin surface for all treated individuals after 28 days of treatment for, on the one hand, a treatment according to the process of the invention (reference 7), and, on the other hand, the placebo treatment (reference 8).

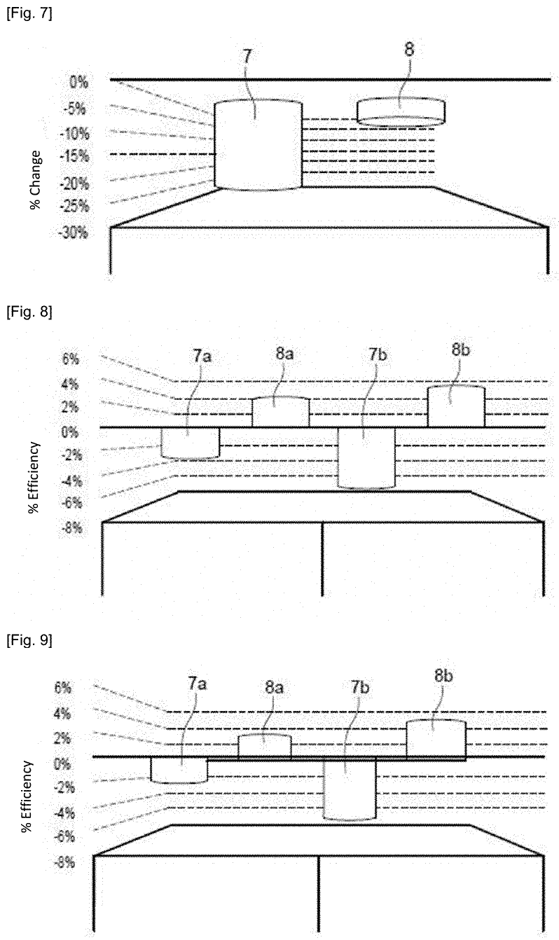

FIG. 9 and FIG. 8 illustrate the results obtained regarding the reduction of wrinkles. The depth of the wrinkles is measured by profilometry. FIGS. 8 and 9 show in histogram form, respectively, the average wrinkle depth (FIG. 8), and the maximum wrinkle depth (FIG. 9) on average for all the individuals treated on the half-face treated via the process of the invention (7a, 7b) and on the half-face treated with the placebo (8a, 8b) at 14 days of treatment (7a, 8a) and at 28 days of treatment (7b, 8b).

It is seen that the placebo worsens the wrinkles by an average of 4%, whereas the treatment of the invention reduces the severity of the wrinkles by an average of 6%.

FIG. 10 illustrates the results obtained by clinical observation on the average percentage of variation in skin smoothing generated by the treatment process of the invention, the references being the same as for FIGS. 8 and 9 and a high percentage reflecting a more substantial smoothing effect. It is seen that the process of the invention (references 7a, 7b) makes it possible to obtain a much greater visible skin smoothing effect than for the placebo (references 8a, 8b), notably at 28 days of treatment (reference 7b).

Finally, some individuals in the study noted an improvement in the uniformity of the complexion, notably by a reduction in the intensity of spots resulting from exposure of the skin to the sun.

The cosmetic treatment process of the invention thus makes it possible to improve the visible appearance of the skin by countering its aging, as demonstrated by the results presented previously, and is an effective antiaging cosmetic treatment.

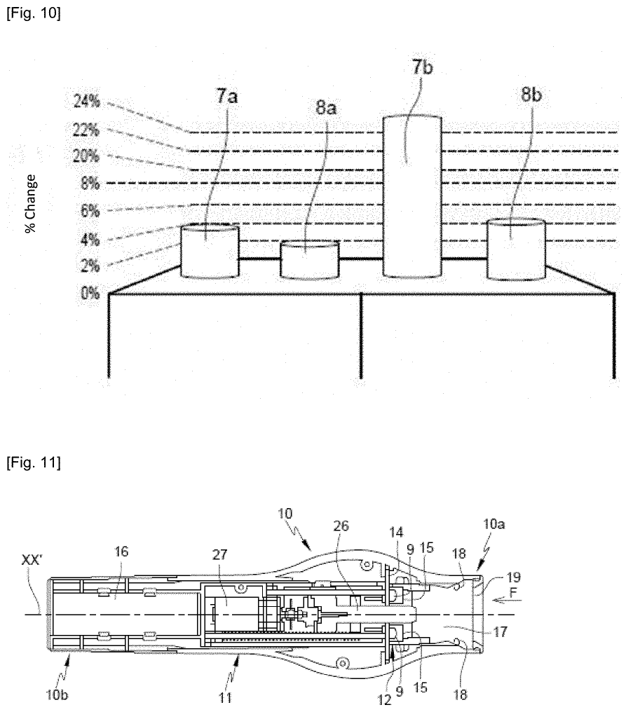

The invention also relates to a device for performing the process of the invention. The device of the invention is described with reference to FIGS. 11 and 12.

The device 10 comprises a case 11 of cylindrical general shape, which includes an electroluminescent device 12 and a removable capsule 13 for applying the composition to the skin. The walls of the device have an embossed part which is exclusively of esthetic nature. The electroluminescent device 12 mainly has a series of light-emitting diodes (LEDs) 9 distributed regularly and circumferentially around the main axis XX′ on the side of a first end 10a of the device 10 and mounted on a dedicated electronic board 14. Each LED 9 is surmounted by a lens 15 forming a light guide and ensuring the focusing of the light emitted by the LEDs 9 in a controlled direction as will be explained hereinbelow. In a preferential embodiment, nine LEDs 9 are arranged circumferentially on the electronic board 14. The LEDs are electrically connected to a battery 16 located in the handle of the device 10 on the side of its opposite end 10b.

With reference to FIG. 12, the device includes, upstream of the LEDs 9 on the side of its first end 10a, a housing 17 for receiving the removable capsule 13. This housing 17 has a frustoconical general shape but may have any suitable shape coinciding with the structure of the outer wall of the capsule 13. Two diametrically opposed hooks 18 are provided in the vicinity of the opening 19 of the housing 17 to ensure the removable attachment of the capsule 13 inserted in the reception housing 17 according to the arrow F.

With reference to FIG. 12, the capsule 13 mainly has a cap 20 that is removable relative to said capsule 13, a dispenser 21 in the form of a ball, a composition reservoir 23 connected to the dispenser 21 by at least one opening 22 ensuring the distribution of the composition onto the surface of the ball 21, and a bottom wall 24 which can slide relative to the ball 21 between a position furthest from the ball 21 in which the composition remains in the reservoir 23, and a position closest to the ball 21 in which the composition, or part of the composition, contained in the reservoir 23 is released onto the surface of the ball 21. The side walls 25 of the capsule 13 are translucent or transparent to ensure that light is scattered over the surface and in the intended direction. More particularly, the lenses 15 associated with the LEDs 13 are configured to ensure the distribution of the emitted light in the walls 25 of the capsule, which are themselves configured to converge the light on the dispensing ball 21, thereby illuminating the area of the skin to which the composition is simultaneously applied. In addition, the dispensing ball 21 is held in place by a flange 28 which forms an extension of the side walls 25 of the capsule, and which is also translucent or transparent. Thus, during illumination, light is distributed on the dispensing ball 21 as indicated previously, but also through the flange 28.

When the capsule 13 is inserted in the reception housing 17, the bottom wall 24 comes into contact with a piston 26 whose axial displacement in the direction of the capsule 13 is ensured by a motor 27 connected to the battery 16. This axial displacement of the piston 26, which is not shown, thus pushes the bottom wall 24 of the capsule 13 toward the ball 21 along a defined path corresponding to the dispensing of a dose of composition, which leads to the release of the composition contained in the reservoir 23 onto the surface of this dispensing ball 21, said ball being intended to roll onto the surface of the skin to be treated and thus to apply the composition onto the skin. At the next use, the piston 26 again drives the bottom wall 24 in axial displacement toward the dispensing ball 21 on a new stroke to deliver a new dose of composition. By way of example, the capsule may be configured to deliver seven doses of composition corresponding to one week's worth of treatment.

A control system not shown is used to control the illumination by the LEDs and the actuation of the piston and the delivery of the composition onto the skin. Preferentially, the illumination and the delivery of the composition are controlled simultaneously.

A person skilled in the art will know how to adapt the device of the invention according to the process of the invention applied. For example, for a composition including niacinamide and illumination at 660 nm, the LEDs 9 or some of the LEDs 9 are configured to emit at 660 nm and the composition contained in the reservoir 23 of the capsule 13 comprises niacinamide. For a process of the invention for which the composition comprises niacinamide and photolyase and illumination at 660 nm and 440 nm, the LEDS 9 or some of the LEDs 9 are configured to emit at 660 nm and 440 nm, respectively, and the composition contained in reservoir 23 comprises niacinamide and photolyase. Finally, when the process of the invention provides for the composition as presented previously in the context of the clinical tests and illumination at 660 nm, 440 nm and 780 nm, the composition contained in reservoir 23 comprises the stated active agents and the LEDS 9 or some of the LEDs 9 are configured to emit at 660 nm and 440 nm, respectively. This is likewise the case when the illumination provides a wavelength of 940 nm.

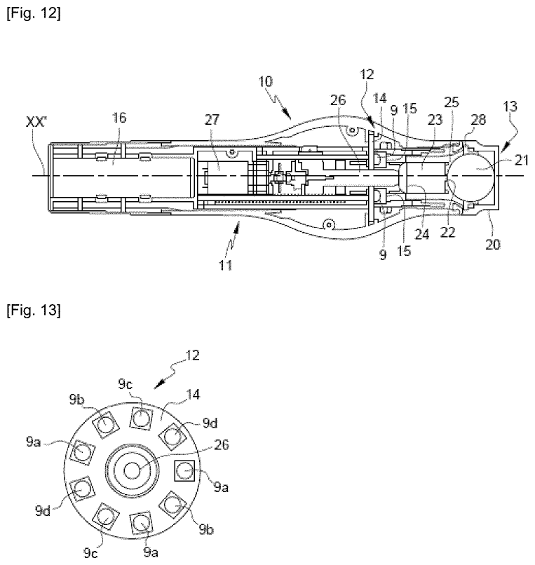

By way of example, FIG. 13 illustrates an embodiment of the electroluminescent device 12 of the invention and more particularly of the assembly of LEDs 9a, 9b, 9c, 9d on the electronic board 14. In this example, there are nine LEDs, distributed circumferentially on the electronic board 14 around the piston 26 which passes through the electronic board 14, it being understood that reference 9a corresponds to the LEDs emitting at 660 nm, reference 9b corresponds to the LEDs emitting at 440 nm, reference 9c corresponds to the LEDs emitting at 940 nm and reference 9d corresponds to the LEDs emitting at 780 nm. The control system not shown may activate all or some of the LEDs, and thus implement either a process according to the invention involving illumination at 660 nm only, or a process according to the invention involving illumination according to one of the three illumination programs shown in Table 2, or else illumination with all four wavelengths mentioned above.

1. A nontherapeutic cosmetic treatment process comprising:
applying to the skin of an individual an active composition comprising at least niacinamide, and
illuminating the skin with at least one light source emitting at a wavelength in a range of from 620 to 690 nanometers,
the illumination being performed before, simultaneously with or after the application of the composition.

2. The process as claimed in claim 1, wherein the active composition also comprises photolyase.

3. The process as claimed in claim 1, wherein the illumination of the skin is also performed with at least one light source emitting at a wavelength in a range of from 410 to 470 nanometers.

4. The process as claimed in claim 3, wherein the illumination of the skin is also performed with at least one light source emitting at a wavelength in a range of from 750 to 810 nanometers.

5. The process as claimed in claim 3, wherein the illumination of the skin is also performed with at least one light source emitting at a wavelength in a range of from 910 to 970 nanometers.

6. The process as claimed in claim 1, wherein a mass percentage of niacinamide in the composition is in a range from 0.5% to 10%.

7. A nontherapeutic cosmetic treatment device comprising:
means for applying to the skin of an individual an active composition comprising at least niacinamide, and
means for illuminating the skin with at least one light source emitting at a wavelength in a range of from 620 to 690 nanometers,
the means for illuminating and applying the active composition being configured so that the illumination can be performed before, simultaneously with or after the application of the composition.

8. The cosmetic treatment device as claimed in claim 7, wherein the active composition also comprises photolyase.

9. The cosmetic treatment device as claimed in claim 7, wherein the means for illuminating the skin also include at least one light source emitting at a wavelength in a range of from 410 to 470 nanometers.

10. The cosmetic treatment device as claimed in claim 7, wherein the means for illuminating the skin also include at least one light source emitting at a wavelength in a range of from 750 to 810 nanometers.

11. The cosmetic treatment device as claimed in claim 7, wherein the means for illuminating the skin also include at least one light source emitting at a wavelength in a range of from 910 to 970 nanometers.

12. The cosmetic treatment device as claimed in claim 7, wherein the at least one light source of the means for illuminating the skin emits at a wavelength of about 660 nanometers.

13. The cosmetic treatment device as claimed in claim 12, wherein the means for illuminating the skin also include at least one light source emitting at a wavelength of about 440 nanometers.

14. The cosmetic treatment device as claimed in claim 13, wherein the means for illuminating the skin also include at least one light source emitting at a wavelength of about 440 nanometers.

15. The cosmetic treatment device as claimed in claim 14, wherein the means for illuminating the skin also include at least one light source emitting at a wavelength of about 780 nanometers.

16. The cosmetic treatment device as claimed in claim 15, wherein the means for illuminating the skin also include at least one light source emitting at a wavelength of about 940 nanometers.

17. The process as claimed in claim 1, wherein the illumination of the skin is performed with at least one light source emitting at a wavelength of about 440 nanometers.

18. The process as claimed in claim 17, wherein the illumination of the skin is also performed with at least one light source emitting at a wavelength of about 440 nanometers.

19. The process as claimed in claim 18, wherein the illumination of the skin is also performed with at least one light source emitting at a wavelength of about 780 nanometers.

20. The process as claimed in claim 19, wherein the illumination of the skin is also performed with at least one light source emitting at a wavelength of about 940 nanometers.
